(19) United States
(12) Patent Application Publication
(10) Pub. No.: US 2024/0247317 A1
(43) Pub. Date: Jul. 25, 2024

(54) Characterising Macrophages and Methods Thereof

(71) Applicant: Agency for Science, Technology and Research, Singapore (SG)

(72) Inventors: Camille Bleriot, Singapore (SG); Florent Ginhoux, Singapore (SG); Ankur Sharma, Singapore (SG)

(21) Appl. No.: 18/040,066

(22) PCT Filed: Aug. 4, 2021
(86) PCT No.: PCT/SG2021/050458

§ 371 Date: Jan. 31, 2023

(30) Foreign Application Priority Data
Aug. 4, 2020 (SG) 10202007446W

(51) Int. Cl. C12Q 1/6886; C12Q 1/6874; C12Q 1/6881

(52) U.S. Cl. CPC C12Q 1/6886; C12Q 1/6881; C12Q 1/6874; C12Q 2600/158

(57) ABSTRACT

There is provided a method of characterising a macrophage in a tissue of a subject, wherein the method comprises determining an expression of one or more biomarkers in the macrophage, wherein the one or more biomarkers is selected from the group consisting of Folr2, SerpinaSi, Nid2, Slc27a6, SemaSa, Cdh13, Bcl6b, C6, Klf15, Marco, Cd209d, Tshz3, Bmpr1a, Tln2, Coro2b, Ackr2, 1110046J04Rik, Pcdhac2, Gm2253, Vsig4, Phactrt, NpM, CpneS, Angptl7, Gm26714, Auts2, Cxcl13, Sgce, 2900052N01 Rik, Cd209b, Timd4, Cd209g, Mrd (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71. In one embodiment, the macrophage can be an embryo-derived macrophage and/or a long-lived tissue resident macrophage. In another embodiment, the macrophage can be a monocyte-derived macrophage. Also disclosed are methods of diagnosing a disease in a subject, methods of treating a disease, and kits for use thereof relating to a macrophage determined to express one or more of the said biomarkers.

### TECHNICAL FIELD

The present disclosure relates broadly to methods of characterising macrophages in a tissue of a subject. The disclosure also relates to a method of classifying or stratifying a disease in a subject in need thereof.

### BACKGROUND

More than 150 years ago, Rudolf Virchow observed that tumours were infiltrated by immune cells that he called a “lymphoreticular infiltrate”. But this observation was a bit lost in time and strategies built on to fight cancer like chemotherapies and radiotherapies did not use much of this biological observation. However, with the emergence of immunotherapies, tumour-associated immune cells are back to the foreground.

The most abundant immune cells in tumour microenvironment (TME) are macrophages (tumour-associated macrophages/TAMs) and a clear positive correlation between their presence in high levels and a bad prognosis for patients is understood in the art. One striking features of macrophages is their high plasticity and the numerous functions that they can play in tissues. Furthermore, origin of macrophages is relatively diverse with a balance between embryonically derived and adult circulating monocyte-derived macrophages that is strongly tissue-dependent.

As such, understanding the pro-tumoral effects of macrophages in an exhaustive manner is still challenging. Accordingly, there is a need to provide a method of characterising a macrophage in a tissue of a subject.

### SUMMARY

In one aspect, there is provided a method of characterising a macrophage in a tissue of a subject, the method comprising determining an expression of one or more biomarkers in the macrophage, wherein the one or more biomarkers is selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chi13, Mir1934, 1810012K08Rik, Ighv1-59, Gm14119, Gm19620 and Snord71.

In some examples, the method further comprises detecting the macrophage in the tissue by selecting cells that express one or more biomarkers selected from the group consisting of CD64, MerTK, Adgre1, CD11b, CD45, CD68, and CD163.

In some examples, the method further comprises determining the characteristics of the cells using t-SNE (T-distributed Stochastic Neighbour Embedding (t-SNE)) and/or UMAP algorithm (Uniform Manifold Approximation and Projection).

In some examples, the macrophage is characterised based on its origin, optionally the method characterises the macrophage is an embryonic-derived macrophage, a long-lived tissue resident macrophage, and/or a monocyte-derived macrophage.

In some examples, the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4 and Cd209g, the macrophage is identified as an embryonic-derived macrophage and/or a long-lived tissue resident macrophage.

In some examples, the macrophage is determined to express one or more Folr2, CD206, CD209, and/or Timd4, the macrophage is identified as an embryonic-derived macrophage and/or a long-lived tissue resident macrophage.

In some examples, the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chi13, Mir1934, 1810012K08Rik, Ighv1-59, Gm14119, Gm19620 and Snord71, the macrophage is identified as a monocyte-derived macrophage.

In another aspect, there is provided a method of diagnosing a disease in a subject in need thereof, wherein the method comprises determining an expression of one or more of biomarkers selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chi13, Mir1934, 1810012K08Rik, Ighv1-59, Gm14119, Gm19620 and Snord71 in a macrophage in a tissue obtained from the subject, wherein the disease is an inflammation, an inflammatory disease, a metabolic disease, or a proliferative disease.

In some examples, the method further comprising a method of providing immunotherapy to the subject based on the expression of the biomarkers.

In some examples, the method characterises the macrophage in the tissue to be one macrophage selected from the group consisting of an embryonic-derived macrophage, a long-lived tissue resident macrophage, and a monocyte-derived macrophage.

In some examples, where the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1 a, TIn2, Coro2b, Ackr2, 1110046J04Rik, Pcdhac2, Gm2253, Vsig4, Phactr1, Npr1, Cpne8, Angptl7, Gm26714, Auts2, Cxc113, Sgce, 2900052N01Rik, Cd209b, Timd4 and Cd209g, the macrophage is identified as an embryonic-derived macrophage and/or a long-lived tissue resident macrophage.

In some examples, where the macrophage is determined to express one or more Folr2, CD206, CD209, and/or Timd4, the macrophage is identified as an embryonic-derived macrophage and/or a long-lived tissue resident macrophage.

In some examples, where the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, the macrophage is identified as a monocyte-derived macrophage.

In some examples, the subject is diagnosed with the disease when: an upregulation of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01Rik, and/or Igsf10 in an embryonic-derived macrophage and/or a long-lived tissue resident macrophage is detected; and/or an upregulation of one or more biomarkers selected from the group consisting of Spp1, IgKv12-38, Ngp, Ighv5-17, Cxcr, Pcdhb16, Pdyn, Ighg3, Igkv6-15, Ighv1-66, Chi13, Ly6c2, Rab44, and/or Ly6i in a monocyte-derived macrophages is detected.

In some examples, the subject is diagnosed with the disease when: an upregulation of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01Rik, and/or Igsf10 in an embryonic-derived macrophage and/or a long-lived tissue resident macrophage is detected; and/or an upregulation of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophage is detected.

In yet another aspect, there is provided a method of treating a disease, comprising modulating a macrophage determined to express one or more biomarkers selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, wherein the disease is an inflammation, an inflammatory disease, a metabolic disease, or a proliferative disease.

In yet another aspect, there is provided a kit for characterising a macrophage, the kit comprising reagents for detecting one or more biomarker selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In some examples, the kit comprises reagents for detecting an embryonic derived macrophage or a long lived tissue resident macrophage comprising one or more reagent for detecting one or more biomarker selected from the group consisting of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, and Cd209g, and/or reagents for detecting a monocyte-derived macrophage comprising one or more reagent for detecting one or more biomarker selected from the group consisting of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

### DESCRIPTION OF EMBODIMENTS

Exemplary, non-limiting embodiments of a method of characterising a macrophage in a tissue of a subject are disclosed hereinafter.

In one aspect, there is provided a method of characterising a macrophage in a tissue of a subject, the method comprising determining an expression of one or more biomarkers in the macrophage, wherein the one or more biomarkers is selected from the group consisting of Folr2, Serpina3i, Nid2, Slc27a6, Sema6a, Cdh13, Bcl6b, C6, Klf15, Marco, Cd209d, Tshz3, Bmpr1 a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

Also provided is a method of characterising a macrophage in a tissue of a subject, the method comprising determining an expression of one or more of biomarkers selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In various embodiments, the method comprises determining and/or detecting and/or quantifying and/or identifying an expression and/or a level of at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, or at least about 51 nucleic acids/genes/proteins/markers in the macrophage.

In various embodiments, the method comprises determining an expression of no more than about 51, no more than about 50, no more than about 49, no more than about 48, no more than about 47, no more than about 46, no more than about 45, no more than about 44, no more than about 43, no more than about 42, no more than about 41, no more than about 40, no more than about 39, no more than about 38, no more than about 37, no more than about 36, no more than about 35, no more than about 34, no more than about 33, no more than about 32, no more than about 31, no more than about 30, no more than about 29, no more than about 28, no more than about 27, no more than about 26, no more than about 25, no more than about 24, no more than about 23, no more than about 22, no more than about 21, no more than about 20, no more than about 19, no more than about 18, no more than about 17, no more than about 16, no more than about 15, no more than about 14, no more than about 13, no more than about 12, no more than about 11, no more than about 10, no more than about nine, no more than about eight, no more than about seven, no more than about six, no more than about five, no more than about four, no more than about three, no more than about two or no more than about one nucleic acid/gene/protein/marker in the macrophage.

To detect/select/sort the macrophages in the tissue of the subject, the methods as disclosed herein may further comprise detecting the macrophage in the tissue by selecting cells that express one or more biomarkers known to be specific to macrophages, such as, but not limited CD64, MerTK, Adgre1, CD11b, CD45, CD68, CD163, and the like. In various embodiments, the method further comprises detecting the macrophage in the tissue by selecting cells that express one or more biomarkers selected from the group consisting of CD64, MerTK, Adgre1, CD11 b, CD45, CD68, and CD163. In some examples, the method further comprises determining the expression of one or more biomarkers selected from the group consisting of CD64, MerTK, Adgre1, CD11 b, CD45, CD68, and CD163.

In various embodiments, the method further comprises determining the characteristics of the cells using t-SNE (T-distributed Stochastic Neighbour Embedding (t-SNE)) and/or UMAP algorithm (Uniform Manifold Approximation and Projection).

In various embodiments, the characterising comprises determining/identifying a type/population (including a subtype/population), an expression profile (for example, at different time points, long term residency of monocyte-derived cell may express the markers of embryonic derived macrophages, such as long-lived tissue resident macrophage), an origin (for example from an embryonic origin/derived, a monocyte origin/derived, and the like), and/or a property (for example an ability/capability and/or propensity to induce/promote inflammation). As such, in various embodiments, the macrophage is characterised based on its origin. For example, the methods as disclosed herein characterises the macrophage to be an embryonic-derived macrophage, a long-lived tissue resident macrophage, and/or a monocyte-derived macrophage.

In various embodiments, the characterising comprises a type or a subtype of the macrophage based on its origin (or where the macrophage was derived from, for example an embryonic-derived macrophage, a long-lived tissue resident macrophage, or a monocyte-derived macrophage).

In various embodiments, the method characterises/detects/sorts/quantify/identify an embryonic-derived macrophage, a long-lived tissue resident macrophage, and/or a monocyte-derived macrophage.

In various embodiments, where the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4 and Cd209g, the macrophage is identified as an embryonic-derived macrophage and/or a long-lived tissue resident macrophage.

In various embodiments, the method comprises determining and/or detecting and/or quantifying and/or identifying an expression and/or a level of at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, or at least about 32 nucleic acids/genes/proteins/markers in the macrophage.

In various embodiments, the method comprises determining an expression of no more than about 32, no more than about 31, no more than about 30, no more than about 29, no more than about 28, no more than about 27, no more than about 26, no more than about 25, no more than about 24, no more than about 23, no more than about 22, no more than about 21, no more than about 20, no more than about 19, no more than about 18, no more than about 17, no more than about 16, no more than about 15, no more than about 14, no more than about 13, no more than about 12, no more than about 11, no more than about 10, no more than about nine, no more than about eight, no more than about seven, no more than about six, no more than about five, no more than about four, no more than about three, no more than about two or no more than about one nucleic acid/gene/protein/marker in the macrophage.

In various embodiments, where the macrophage is determined to express one or more Folr2, CD206, CD209, and/or Timd4, the macrophage is identified as an embryonic-derived macrophage and/or a long-lived tissue resident macrophage.

In various embodiments, where the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, the macrophage is identified as a monocyte-derived macrophage.

In various embodiments, the method comprises determining and/or detecting and/or quantifying and/or identifying an expression and/or a level of at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, or at least about 19 nucleic acids/genes/proteins/markers in the macrophage.

In various embodiments, the method comprises determining an expression of no more than about 19, no more than about 18, no more than about 17, no more than about 16, no more than about 15, no more than about 14, no more than about 13, no more than about 12, no more than about 11, no more than about 10, no more than about nine, no more than about eight, no more than about seven, no more than about six, no more than about five, no more than about four, no more than about three, no more than about two or no more than about one nucleic acid/gene/protein/marker in the macrophage.

Also disclosed is a method of classifying/stratification/diagnose/prognose a disease in a subject in need thereof, wherein the method comprises determining an expression of one or more of biomarkers selected from the group consisting of Folr2, Serpina3i, Nid2, Slc27a6, Sema6a, Cdh13, Bcl6b, C6, Klf15, Marco, Cd209d, Tshz3, Bmpr1 a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71 in a macrophage obtained from the subject.

Thus, in another aspect, there is provided a method of diagnosing a disease in a subject in need thereof, wherein the method comprises determining an expression of one or more of biomarkers selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71 in a macrophage in a tissue obtained from the subject, wherein the disease is an inflammation, an inflammatory disease, a metabolic disease, or a proliferative disease.

In various embodiments, the macrophage is obtained/sorted/detected in a tissue (such as a biopsy) of the subject.

In various embodiments, the method further comprising a method of providing immunotherapy to the subject based on the expression of the biomarkers.

In some examples, the method further comprises administering to the subject in need thereof an agent capable of targeting (such as modulating (or inhibiting or activating) the function of) embryonic-derived macrophages and/or long-lived tissue resident macrophages and/or an agent capable of targeting (such as modulating (or inhibiting or activating) the function of) monocyte-derived macrophages. In some examples, the method further comprises administering to the subject in need thereof an agent capable of targeting embryonic-derived macrophages or long-lived tissue resident macrophages and/or an agent capable of targeting (such as modulating (or inhibiting) the function of) monocyte-derived macrophages. In some examples, the agent may be an antibody or fragment thereof.

In various embodiments, the method characterises/detects/sorts/quantify/identify an embryonic-derived macrophage and/or a long-lived tissue resident macrophage and/or a monocyte-derived macrophage.

In various embodiments, where the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4 and Cd209g, the macrophage is identified as an embryonic-derived macrophage and/or a long-lived tissue resident macrophage.

In various embodiments, the method comprises determining and/or detecting and/or quantifying and/or identifying an expression and/or a level of at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, or at least about 32 nucleic acids/genes/proteins/markers in the macrophage.

In various embodiments, the method comprises determining an expression of no more than about 32, no more than about 31, no more than about 30, no more than about 29, no more than about 28, no more than about 27, no more than about 26, no more than about 25, no more than about 24, no more than about 23, no more than about 22, no more than about 21, no more than about 20, no more than about 19, no more than about 18, no more than about 17, no more than about 16, no more than about 15, no more than about 14, no more than about 13, no more than about 12, no more than about 11, no more than about 10, no more than about nine, no more than about eight, no more than about seven, no more than about six, no more than about five, no more than about four, no more than about three, no more than about two or no more than about one nucleic acid/gene/protein/marker in the macrophage.

In various embodiments, where the macrophage is determined to express one or more Folr2, CD206, CD209, and/or Timd4, the macrophage is identified as an embryonic-derived macrophage and/or a long-lived tissue resident macrophage.

In various embodiments, where the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, the macrophage is identified as a monocyte-derived macrophage.

In various embodiments, the method comprises determining and/or detecting and/or quantifying and/or identifying an expression and/or a level of at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, or at least about 19 nucleic acids/genes/proteins/markers in the macrophage.

In various embodiments, the method comprises determining an expression of no more than about 19, no more than about 18, no more than about 17, no more than about 16, no more than about 15, no more than about 14, no more than about 13, no more than about 12, no more than about 11, no more than about 10, no more than about nine, no more than about eight, no more than about seven, no more than about six, no more than about five, no more than about four, no more than about three, no more than about two or no more than about one nucleic acid/gene/protein/marker in the macrophage.

In various embodiments, the subject is classified/stratified/diagnosed/prognosed with the disease (for example cancer such as pancreatic adenocarcinoma) when:
an upregulation/increase of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01 Rik, and/or Igsf10 in an embryonic-derived macrophage and/or a long-lived tissue resident macrophage is observed/determined/detected/identified; and/or
an upregulation/increase of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophages is observed/determined/detected/identified.

In various embodiments, the subject is diagnosed with the disease when: an upregulation of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01 Rik, and/or Igsf10 in an embryonic-derived macrophage and/or a long-lived tissue resident macrophage is detected; and/or an upregulation of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophages is detected.

In various embodiments, the subject is classified/stratified/diagnosed/prognosed with the disease (for example cancer such as pancreatic adenocarcinoma, melanoma, and/or breast cancer) when:
an upregulation/increase of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01 Rik, and/or Igsf10 in an embryonic-derived macrophage and/or a long-lived tissue resident macrophage is observed/determined/detected/identified; and/or
an upregulation/increase of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophage is observed/determined/detected/identified.

In yet another aspect, there is provided a method of treating a disease, comprising modulating a macrophage determined to express one or more biomarkers selected from the group consisting of Folr2, Serpina3i, Nid2, Slc27a6, Sema6a, Cdh13, Bcl6b, C6, Klf15, Marco, Cd209d, Tshz3, Bmpr1 a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In various embodiments, the method further comprises characterising/detecting/sorting/quantifying/identifying an embryonic-derived macrophage, a long-lived tissue resident macrophage, and/or a monocyte-derived macrophage.

In various embodiments, the modulation is increasing or decreasing the expression of the biomarkers, and/or increasing or decreasing the activity/proliferation/migration of an embryonic-derived macrophage, a long-lived tissue resident macrophage, and/or a monocyte-derived macrophage, and/or modulating/regulating/inhibiting/activating one or more other immune cells functions (such as B cell function and/or T cell function), and/or modulating/regulating/inhibiting one or more cancer cell function/phenotype/response to a drug, and/or modulating/regulating/inhibiting one or more stromal (such as fibroblast/endothelial) cell function/phenotype.

In various embodiments, the macrophage is a tumour-associated macrophage and/or an inflammation site-associated macrophage.

In various embodiments, the subject is a human and/or animal such as a non-human primate, a rodent (such as a mouse, a rat, a rabbit, a guinea pig, and the like), and the like.

In various embodiments, the subject is a human suspected of or is suffering from a medical condition (or disease).

In various embodiments, the disease is an inflammation, an inflammatory disease, a metabolic disease, or a proliferative disease.

In various embodiments, the inflammatory disease is a liver cirrhosis.

In various embodiments, disease is a proliferative disease such as tumour and/or cancer.

In various embodiments, the metabolic disease may include non-alcoholic steatohepatitis (NASH) and obesity-related disorders.

In various embodiments, the disease is a cancer such as, but is not limited to, liver cancer (such as hepatocellular carcinoma), bone cancer, pancreatic cancer (such as pancreatic ductal adenocarcinoma (PDAC), pancreatic adenocarcinoma, and the like), skin cancer (such as melanoma, including but not limited to, cutaneous or intraocular malignant melanoma), cancer of the head or neck, breast cancer, lung cancer, renal cancer, uterine cancer, bowel cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukaemia, chronic lymphoid leukaemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukaemia, mycosis fungoides, anaplastic large cell lymphoma, T-cell lymphoma, and precursor T-lymphoblastic lymphoma, and any combinations of cancers thereof.

In various embodiments, the disease is a proliferative disease such as tumour and/or cancer such as, but is not limited to liver tumour, pancreatic tumour, hepatocellular carcinoma, pancreatic ductal adenocarcinoma (PDAC), pancreatic adenocarcinoma, melanoma, breast cancer, and the like.

In various embodiments, the tissue comprises an inflammation site and/or a tumour and/or a cancerous growth.

Also disclosed are kits/biomarker panels for characterising a macrophage, a disease (such as an inflammation and/or inflammatory disease such as tumour and/or cancer), the kit/biomarker panel comprising one or more reagent for detecting a biomarker selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

Also disclosed are kits/biomarker panels for characterising an embryonic-derived macrophage or a long-lived tissue resident macrophage in a disease (such as an inflammation and/or inflammatory disease such as tumour and/or cancer), the kit/biomarker panel comprising one or more reagent for detecting a biomarker selected from the group consisting of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01Rik, Cd209b, Timd4, and Cd209g.

Also disclosed are kits/biomarker panels for characterising a monocyte-derived macrophage in a disease (such as an inflammation and/or inflammatory disease such as tumour and/or cancer), the kit/biomarker panel comprising one or more reagent for detecting a biomarker selected from the group consisting of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In yet another aspect, there is provided a kit for characterising a macrophage, the kit comprising reagents for detecting one or more biomarker selected from the group consisting of Folr2, Serpina3i, Nid2, Slc27a6, Sema6a, Cdh13, Bcl6b, C6, Klf15, Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In various embodiments, the kit comprises reagents for detecting an embryonic derived macrophage or a long lived tissue resident macrophage comprising one or more reagent for detecting one or more biomarker selected from the group consisting of Folr2, Serpina3i, Nid2, Slc27a6, Sema6a, Cdh13, Bcl6b, C6, Klf15, Mrc1 (CD206), Marco, Cd209d, Tshz3, Bmpr1a, TIn2, Coro2b, Ackr2, 1110046J04Rik, Pcdhac2, Gm2253, Vsig4, Phactr1, Npr1, Cpne8, AngptI7, Gm26714, Auts2, Cxcl13, Sgce, 2900052N01 Rik, Cd209b, Timd4, and Cd209g, and/or reagents for detecting a monocyte-derived macrophage comprising one or more reagent for detecting one or more biomarker selected from the group consisting of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

In various embodiments, the macrophage is a tumour-associated macrophage and/or inflammation-associated macrophage.

In various embodiments, the macrophages as described herein may be used as a therapy or a medicine.

The term “associated” may be used interchangeably with “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible.

The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications or changes may be made without deviating from the scope of the invention. Example embodiments are not necessarily mutually exclusive as some may be combined with one or more embodiments to form new exemplary embodiments. The example embodiments should not be construed as limiting the scope of the disclosure.

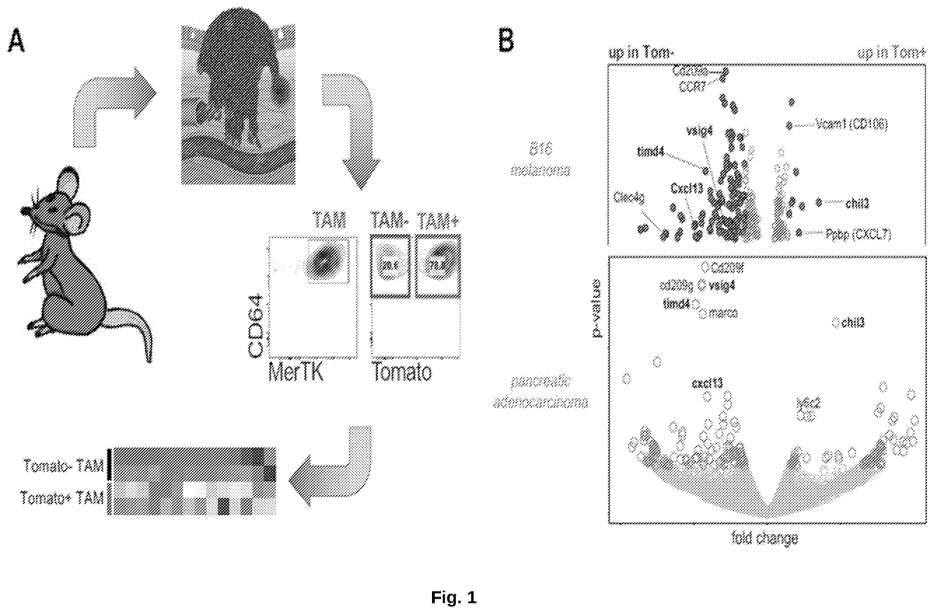

FIG. 1 shows a (A) diagram showing the strategy as described herein. The ms4a3cre-RosaTdt mice are transplanted with tumour cells and tumour-associated macrophages either from embryonic origin (TAM−) or monocyte-derived (TAM+) were FACS-sorted. RNA is then extracted and transcriptomic signatures of both populations were generated and compared. (B) Volcano plots of the genes differentially expressed in TAM- and TAM+ macrophages showing common trends across tissues.

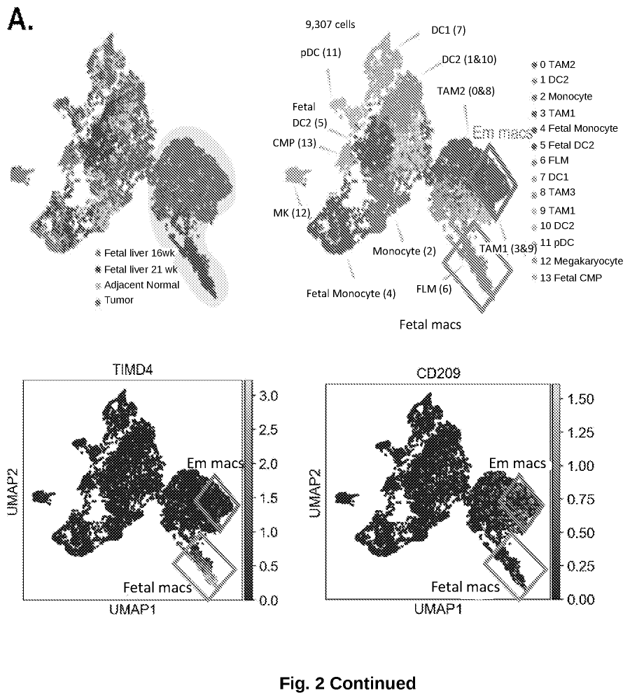

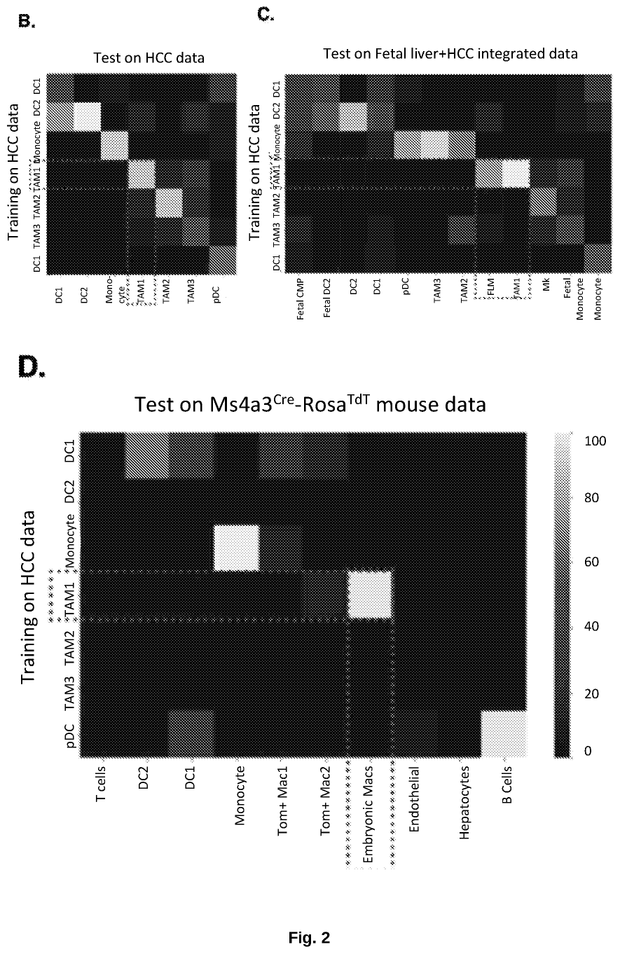

FIG. 2 shows analysis of human HCC dataset (A) Integrated myeloid cells from foetus, normal and tumour tissues. Within TAM (pink cluster on the left panel), a subpopulation displaying similarities with foetal macrophages (in green) can be identified (TAM1 or Em macs in red). Cells from this cluster express for example genes such as Timd4 and Cd209. Machine learning based (B) Correlation heatmap comparing the HCC training on HCC test. Note the high correlation scores as proof-of-principle high correlation scores. (C) Correlation heatmap comparing the HCC training on Foetal liver and HCC combined dataset. Note the high correlation between HCC TAM1 and foetal-liver macrophage. (D) Correlation heatmap comparing the HCC training on mouse dataset. Note in fate mapper Ms4a3^{Cre}-Rosa^{TdT} mice Tom (tomato) positive cells are of monocyte origin while Tom negative macrophages are embryonic origin. Note the high correlation between TAM1 and mouse embryonic (Tom negative) macrophages.

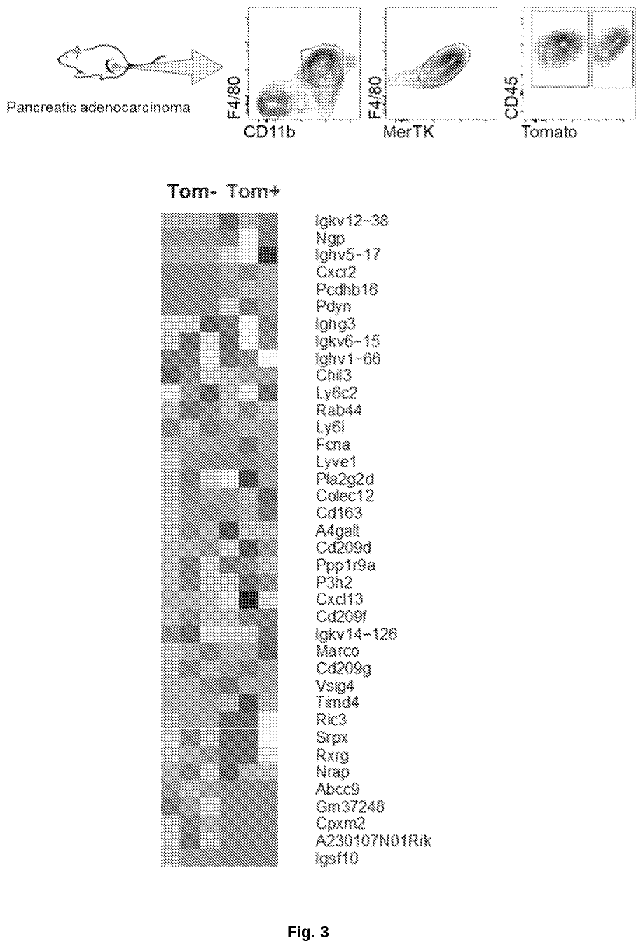

FIG. 3 shows the strategy and generation of top genes differentially expressed in Em- and Mo- TAMs in mouse pancreatic cancer model.

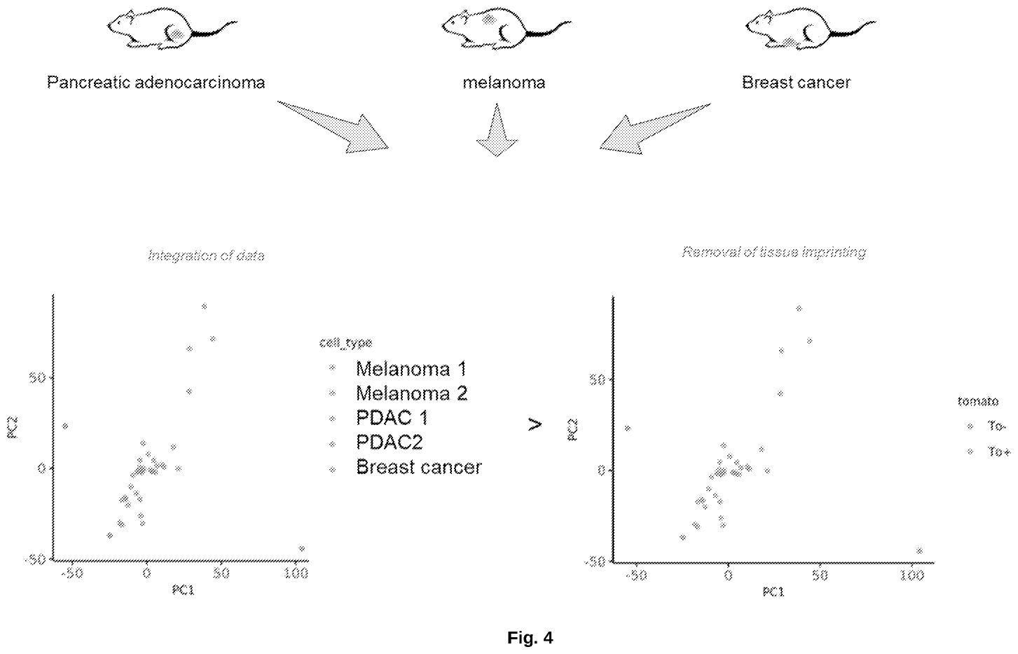

FIG. 4 shows the extension of the strategy as disclosed herein by defining DEG in Em- and Mo-TAMs in melanoma and breast cancer.

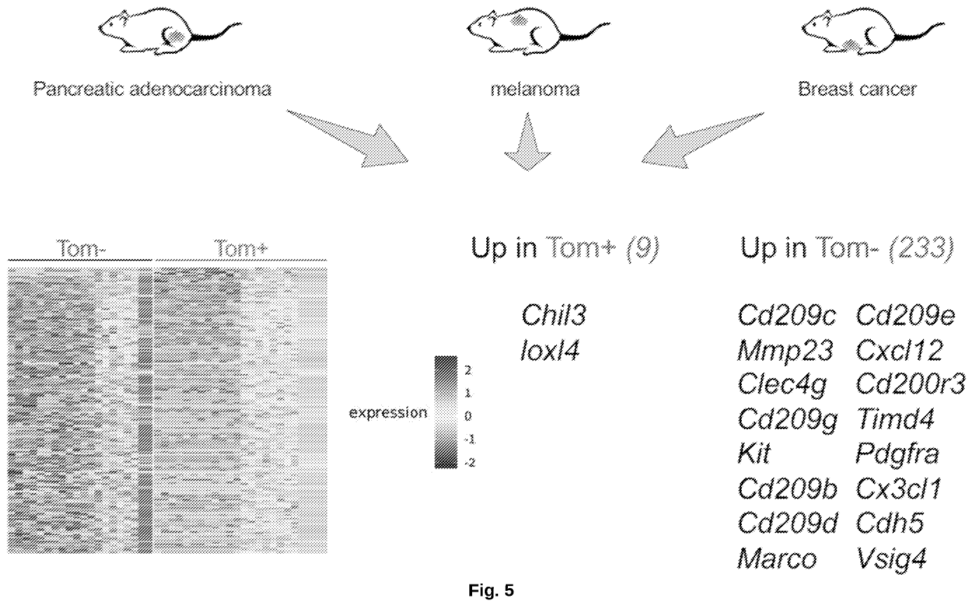

FIG. 5 shows a shortlist of DEGs conserved in all the models of mouse cancers tested. IN addition of this “core Em- vs. Mo- TAM” DEG, we could identify DEGs specifically in each tissue context.

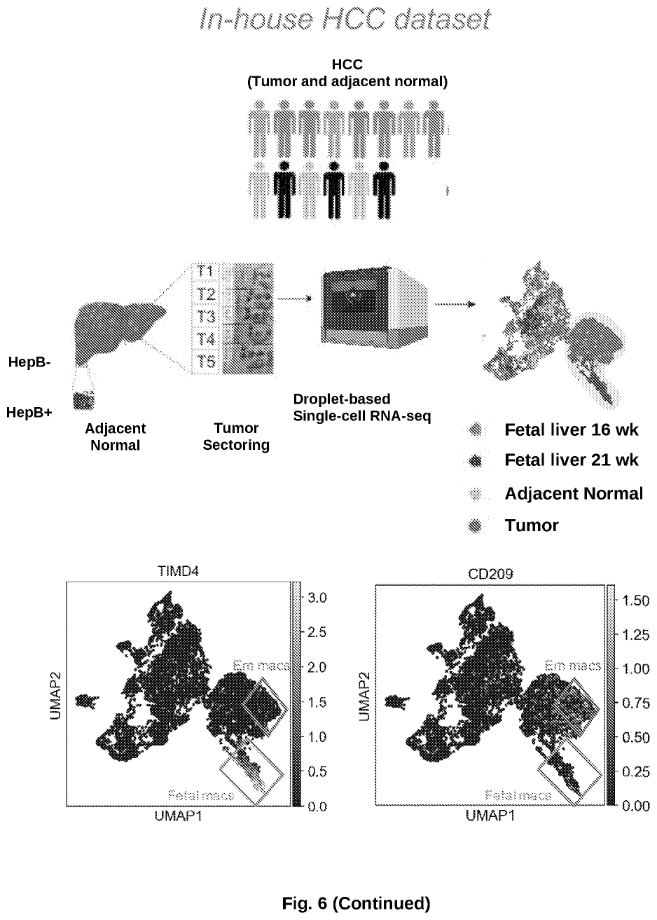

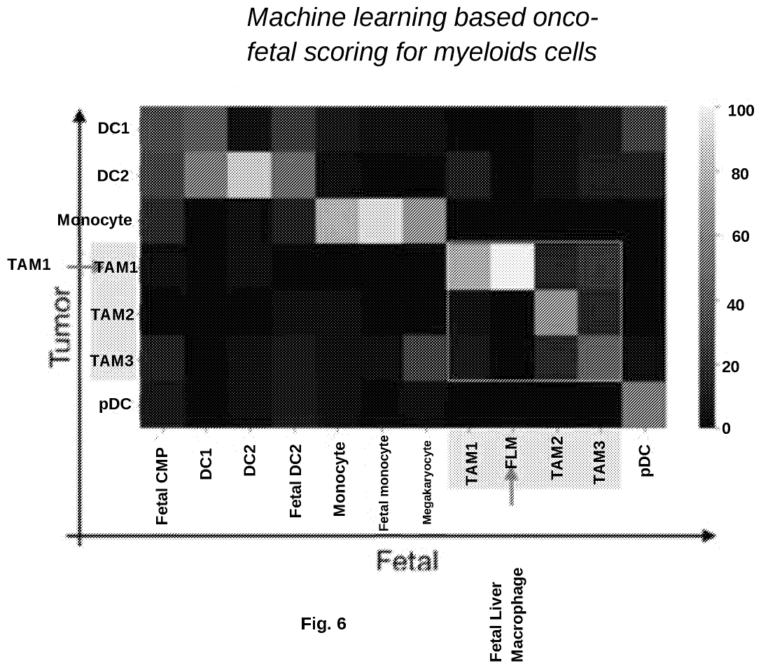

FIG. 6 shows the mapping of embryonic and monocyte-derived human TAMs. This figure shows TAM can be tracked in human cancer.

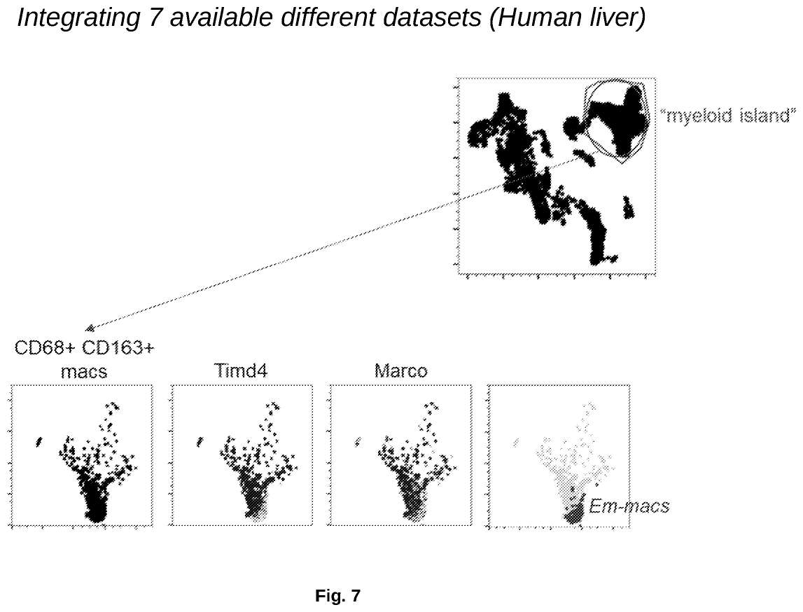

FIG. 7 shows the validation of the mapping of embryonic and monocyte-derived human TAMs by integrating seven different datasets (human liver).

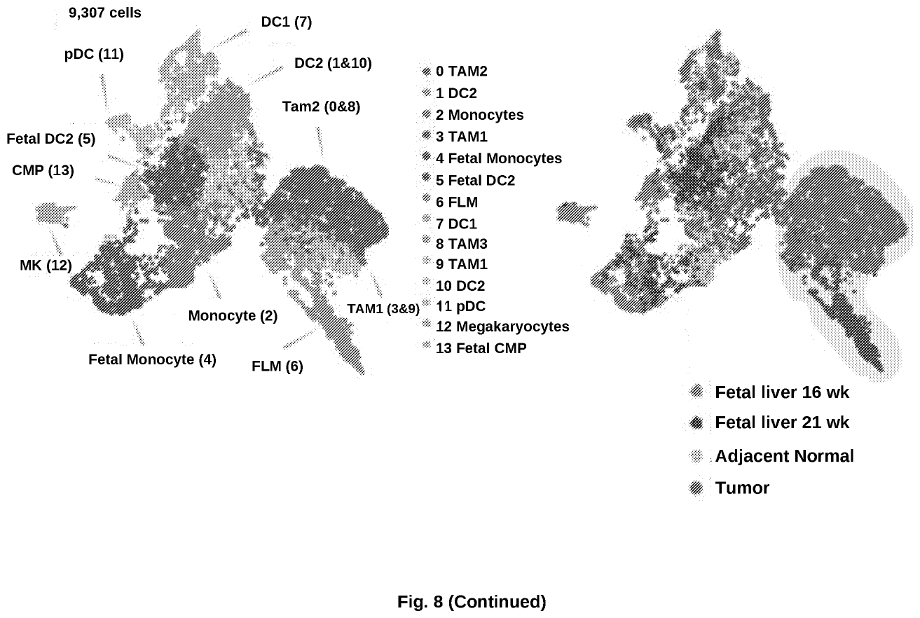

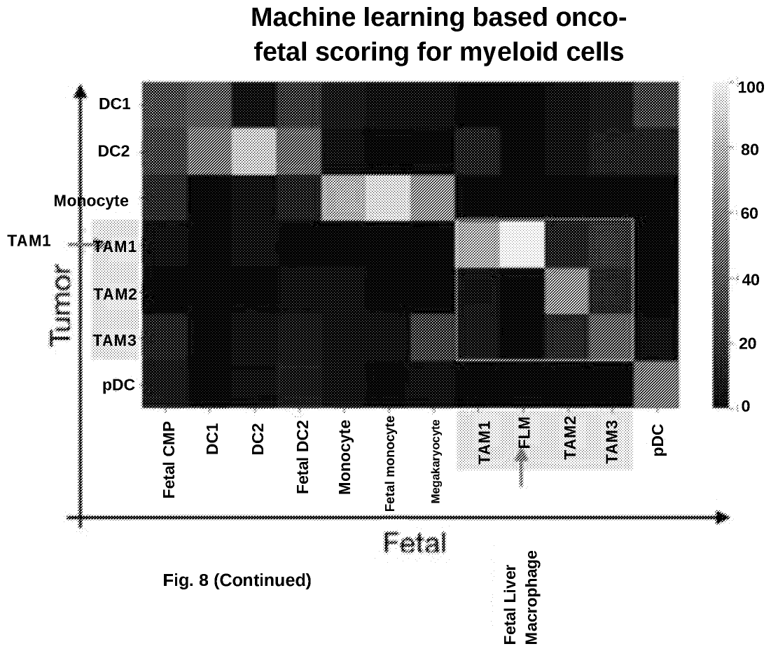

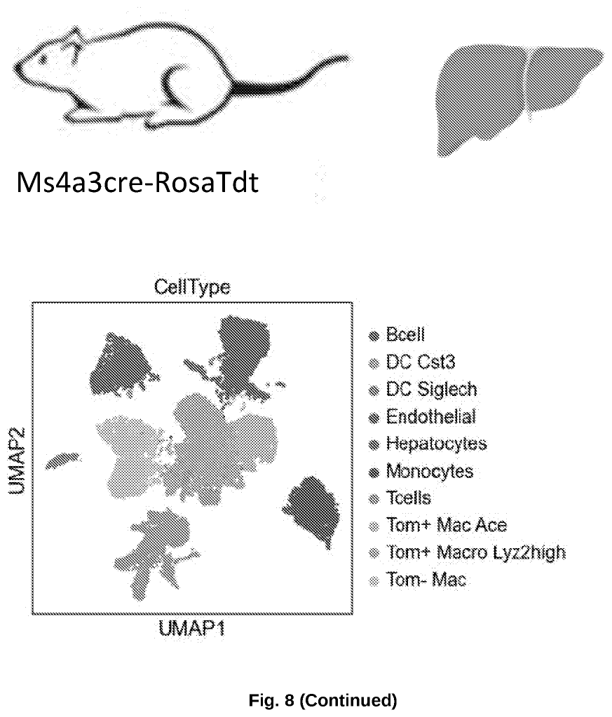

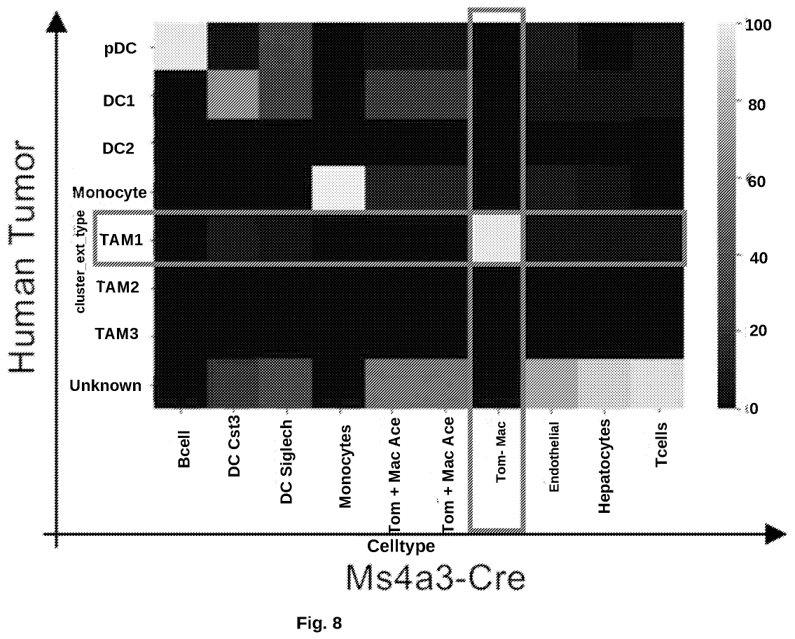

FIG. 8 shows the comparison of human and mouse Em- and Mo- TAMs. Quality control of mouse/human liver signatures.

### EXPERIMENTAL SECTION

The inventors of the present disclosure had developed a new mouse model (Ms4a3cre-RosaTdt mice) that can clearly distinguish embryonically derived vs. monocyte derived macrophages (Liu et al., 2019, Fate mapping via Ms4a3-expression history traces monocyte-derived cells. Cell 178, 1509-1525). The inventors of the present disclosure used two different transplantable and one genetic model of tumours in this new murine model to generate accurate transcriptomic signatures of embryonic- (Em-) vs. monocyte-derived (Mo-) TAMs (FIG. 1). Several markers for each population was distinguished for diagnosis in humans.

Embryonic-derived TAM biomarkers: Serpina3i, Nid2, Slc27a6, Sema6a, Cdh13, Bcl6b, C6, Klf15, Marco, Cd209d, Tshz3, Bmpr1 a, TIn2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01 Rik, Cd209b, Timd4, Cd209g.

Monocyte-derived TAMs biomarkers: 1122ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620, Snord71.

To identify signatures to track Em- and Mo-TAMs, machine learning based approach has been employed. A large dataset of macrophage populations was generated from human foetal liver and hepatocellular carcinoma patients (HCC) (FIG. 2). Here three sub-populations of TAMs were identified among which TAM1 displayed similarities with human foetal macrophages (FLM) and mouse embryonic macrophages. Interestingly, conservation of genes (FOLR2, CD206, CD209) was observed between mouse embryonic, human foetal liver and HCC TAM1 populations. Similar populations in inflammatory conditions such as liver cirrhosis was also identified. Given the similarities between cancer and inflammation the strategy of targeting macrophage ontogeny as described herein will have implications beyond cancers. These observations are also validated in other published datasets of HCC (Zhang et al., 2019) and human pancreatic ductal adenocarcinoma (PDAC) (Peng et al., 2019).

These findings is also validated by generating single-cell RNA-seq datasets using the new cutting-edge BD Rhapsody technology that allows the screening of a selected panels of ˜400 genes in thousands of cells and several samples from distinct tumours contexts (liver, pancreas, melanoma and breast cancers). The inventors of the present disclosure have designed the panel by focusing on the signatures of Em- and Mo- TAMs and this toolbox will allow the generation of relevant data quickly and efficiently to confirm preliminary observations.

The dichotomy between Em- and Mo- macrophages is a feature that is not only observed in cancer but in virtually all diseases for which macrophages are involved including inflammatory diseases such as liver cirrhosis for example.

### Applications

Embodiments of the methods disclosed herein provide a fast, efficient and thorough method of characterising macrophages found in the tissue of a subject.

Advantageously, the methods disclosed can identify subpopulation of macrophages. For example, the methods as disclosed herein can identify subpopulations having different origins.

Even more advantageously, the biomarkers as disclosed herein are capable of differentiating embryonic- and monocyte- derived tumour associated macrophages. The transcriptional signatures of the embryonic- and monocyte-macrophages and/or embryonic- and adult (long-lived tissue resident)- macrophage score can advantageously provide an indication of tumour regression or progression.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of characterising a macrophage in a tissue of a subject, the method comprising:
determining an expression of one or more biomarkers in the macrophage, wherein the one or more biomarkers is selected from the group consisting of Folr2, Serpina3i, Nid2, Slc27a6, Sema6a, Cdh13, Bcl6b, C6, Klf15, Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71,
wherein the method characterises the macrophage as being an embryonic-derived macrophage, a long-lived tissue resident macrophage, and/or a monocyte-derived macrophage.

2. The method according to claim 1, wherein the method further comprises detecting the macrophage in the tissue by selecting cells that express one or more biomarkers selected from the group consisting of CD64, MerTK, Adgre1, CD11b, CD45, CD68, and CD163.

3. The method according to claim 1, wherein the method further comprises determining the characteristics of the cells using t-SNE (T-distributed Stochastic Neighbour Embedding (t-SNE)) and/or UMAP algorithm (Uniform Manifold Approximation and Projection).

4. The method according to claim 1, wherein the macrophage is characterised based on its origin.

5. The method according to a claim 1, wherein where the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4 and Cd209g, the macrophage is identified as an embryonic-derived macrophage and/or a long-lived tissue resident macrophage.

6. The method according to claim 1, wherein where the macrophage is determined to express one or more Folr2, CD206, CD209, and/or Timd4, the macrophage is identified as an embryonic-derived macrophage and/or a long-lived tissue resident macrophage.

7. The method according to claim 1, wherein where the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, the macrophage is identified as a monocyte-derived macrophage.

8. A method of treating a disease in a subject in need thereof,
wherein the method comprises:
a) diagnosing a disease in a subject in need thereof, wherein the diagnosing said disease in the subject comprises:
i) determining an expression of one or more of biomarkers selected from the group consisting of Folr2, Serpina3i, Nid2, Slc27a6, Sema6a, Cdh13, Bcl6b, C6, Klf15, Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71 in a macrophage in a tissue obtained from the subject,
characterising the macrophage to be an embryonic-derived macrophage, a long-lived tissue resident macrophage, and/or a monocyte-derived macrophage,
ii) determining differential expression of biomarkers in the tissue, as compared to a control in order to determine the subject to have said disease; and
b) treating the subject determined to have the disease,
wherein the disease is an inflammation, an inflammatory disease, a metabolic disease, or a proliferative disease.

9. The method according to claim 8, wherein the method further comprising a method of providing immunotherapy to the subject based on the expression of the biomarkers.

10. (canceled)

11. The method according to claim 8, wherein where the macrophage is determined to express one or more of Folr2, Serpina3i, Nid2, Slc27a6, Sema6a, Cdh13, Bcl6b, C6, Klf15, Mrc1 (CD206), Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4 and Cd209g, the macrophage is identified as an embryonic-derived macrophage and/or a long-lived tissue resident macrophage.

12. The method according to claim 8, wherein where the macrophage is determined to express one or more Folr2, CD206, CD209, and/or Timd4, the macrophage is identified as an embryonic-derived macrophage and/or a long-lived tissue resident macrophage.

13. The method according to claim 8, wherein where the macrophage is determined to express one or more of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, the macrophage is identified as a monocyte-derived macrophage.

14. The method according to claim 8, wherein the subject is diagnosed with the disease when:
an upregulation of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01Rik, and/or Igsf10 in an embryonic-derived macrophage and/or a long-lived tissue resident macrophage is detected; and/or
an upregulation of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophages is detected.

15. The method according to claim 8, wherein the subject is diagnosed with the disease when:
an upregulation of one or more biomarkers selected from the group consisting of Folr2, Mrc1 (CD206), Marco, Vsig4, Fcna, Lyve1, Pla2g2d, Colec12, Cd163, A4galt, Cd209d, Ppp1r9a, P3h2, Cxcl13, Cd209f, Igkv 14-126, Cd209g, Timd4, Ric3, Srpx, Rxrg, Nrap, Abcc9, Gm37248, Cpxm2, A230107N01Rik, and/or Igsf10 in an embryonic-derived macrophage and/or a long-lived tissue resident macrophage is detected; and/or
an upregulation of one or more biomarkers selected from the group consisting of Spp1, IgKv12-38, Ngp, Ighv5-17, Cxcr, Pcdhb16, Pdyn, Ighg3, Igkv6-15, Ighv1-66, Chil3, Ly6c2, Rab44, and/or Ly6i in a monocyte-derived macrophage is detected.

16. The method according to claim 8, wherein the method further comprises:
modulating a macrophage determined to express one or more biomarkers selected from the group consisting of Folr2, Serpina3i, Nid2, Slc27a6, Sema6a, Cdh13, Bcl6b, C6, Klf15, Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.

17. A kit for characterising a macrophage, the kit comprising reagents for detecting one or more biomarker selected from the group consisting of Folr2, Serpina3i, Nid2, Slc27a6, Sema6a, Cdh13, Bcl6b, C6, Klf15, Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4, Cd209g, Mrc1 (CD206), Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71, wherein the kit characterises the macrophage as being an embryonic-derived macrophage, a long-lived tissue resident macrophage, and/or a monocyte-derived macrophage.

18. The kit of claim 17, wherein the kit comprises
reagents for detecting an embryonic derived macrophage or a long lived tissue resident macrophage comprising one or more reagent for detecting one or more biomarker selected from the group consisting of Folr2, Serpina3i, Nid2, Slc27a6, Sema6a, Cdh13, Bcl6b, C6, Klf15, Mrc1 (CD206), Marco, Cd209d, Tshz3, Bmpr1a, Tln2, Coro2b, Ackr2, 1110046J04Rik, Pcdhac2, Gm2253, Vsig4, Phactr1, Npr1, Cpne8, Angptl7, Gm26714, Auts2, Cxcl13, Sgce, 2900052N01Rik, Cd209b, Timd4, and Cd209g, and/or
reagents for detecting a monocyte-derived macrophage comprising one or more reagent for detecting one or more biomarker selected from the group consisting of Spp1, Il22ra2, Gm24112, Gm23010, Ighv7-1, Gm23628, Gm24620, Gm23058, Arhgef37, Gad1-ps, Gm26397, Ighv2-5, Chil3, Mir1934, 1810012K08Rik, Ighv1-59, Gm14119, Gm19620 and Snord71.
